(19) United States
(12) Patent Application Publication
(10) Pub. No.: US 2021/0380695 A1
(43) Pub. Date: Dec. 9, 2021

(54) STING AGONIST COMBINATION TREATMENTS WITH IMMUNE CHECKPOINT INHIBITORS

(71) Applicants: ImmuneSensor Therapeutics, Inc., Dallas, TX (US); The Board of Regents of the University of Texas System, Austin, TX (US)

(72) Inventors: Zhijian CHEN, Dallas, TX (US); Lijun SUN, Dallas, TX (US)

(73) Assignees: ImmuneSensor Therapeutics, Inc., Dallas, TX (US); The Board of Regents of the University of Texas System, Austin, TX (US)

(21) Appl. No.: 17/322,827

(22) Filed: May 17, 2021

Related U.S. Application Data
(60) Provisional application No. 63/091,874, filed on Oct. 14, 2020.
(60) Provisional application No. 63/025,905, filed on May 15, 2020.

(51) Int. Cl. C07K 16/28; A61K 31/7084; A61K 47/68; A61P 35/00

(52) U.S. Cl. CPC C07K 16/2818; A61K 31/7084; A61K 2039/545; A61P 35/00; C07K 16/2827; A61K 47/6807

(57) ABSTRACT

The disclosure provides, among other things, methods and uses for treating a disease or disorder, particularly a cancer, in a patient, comprising conjointly administering a CTLA4 inhibitor (e.g., an anti-CTLA4 antibody) and a STING agonist to the patient, wherein the CTLA4 inhibitor is administered intratumorally to the patient. The STING agonist can be administered intratumorally, orally or systemically (e.g., intravenously, intramuscularly, or subcutaneously) to the patient.

### 1. FIELD

This disclosure pertains to, among other things, the use of an intratumorally administered antibody against cytotoxic T-lymphocyte-associated protein 4 (CTLA4) in combination with a STING agonist for activating the immune system to treat certain diseases or disorders, including cancer.

### 2. BACKGROUND

The treatment of advanced solid tumor malignancies as well as many hematologic malignancies continues to be defined by high unmet medical need. In most settings, treatment with cytotoxic chemotherapy and targeted kinase inhibitors leads to the emergence of drug-resistant tumor clones and subsequent tumor progression and metastasis.

In recent years, notable success has been achieved through alternate approaches oriented around activation of immune-mediated tumor destruction. The immune system plays a pivotal role in defending humans and animals against cancer. The anti-tumor effect is controlled by positive factors that activate anti-tumor immunity and negative factors that inhibit the immune system. Negative factors that inhibit anti-tumor immunity include immune checkpoint proteins, such as cytotoxic T-lymphocyte-associated protein 4 (CTLA4), programmed cell death-1 (PD-1), and programmed death-ligand 1 (PD-L1). Immuno-oncology (TO) approaches, including antibodies against these checkpoint proteins, have shown remarkable efficacy in several types of human cancers.

However, existing cancer immunotherapy through immune checkpoint blockade is effective for only a small fraction (on average 20-30%) of cancer patients. The patients who are refractory to immune checkpoint blockade often have tumors that are not inflamed, or so-called “cold” tumor cells, i.e., they lack tumor-infiltrating leukocytes (TILs), such as cluster of differentiation 8 (CD8) T cells, or the tumor microenvironment suppresses the functions of the TILs. A major thrust of ongoing cancer drug development research remains focused on transforming “cold” tumor cells into “hot” tumor cells in order to achieve better tumor control across a wider array of patients.

The innate immune system, which is the first line of defense against pathogens and cancer cells, is important for turning the non-inflamed tumors (“cold”) into an inflamed (“hot”) microenvironment. A recently discovered innate immunity pathway, the Cyclic GMP-AMP Synthase (cGAS)-Stimulator of Interferon Genes (STING) pathway, plays a critical role in anti-tumor immunity. cGAS is a DNA sensing enzyme that activates the type-I interferon pathway. Upon binding to DNA, cGAS is activated to synthesize 2′3′-cyclic-GMP-AMP (2′3′-cGAMP), which then functions as a secondary messenger that binds to and activates the adaptor protein STING. STING then activates a signal transduction cascade leading to the production of type-I interferons, cytokines and other immune mediators.

While cytokine production is essential for generating anti-tumor immunity, high cytokines levels pose a safety concern. Specifically, high cytokine levels can evoke an inflammatory response in cancer patients undergoing immunotherapy. The inflammatory response can be enhanced in the presence of other compounds that modulate the immune system, for instance, immune checkpoint inhibitors. Developing immunotherapies with improved therapeutic indexes remains a high priority.

Administration of anti-CTLA4 antibodies is often associated with severe auto-immune toxicity. See Frasen et al. Clin. Cancer Res. 19:5831-5839 (2013). Prior studies have shown that administration of low doses of anti-CTLA4 antibodies administered locally at the site of the tumor can potentially overcome some of the toxicological problems associated with systemic administration of anti-CTLA4 antibodies at higher doses. However, the local administration of low doses of anti-CTLA4 antibodies at the site of the tumor may suffer from insufficient efficacy. Therefore, developing highly efficacious, toxicologically acceptable methods to administer anti-CTLA4 antibodies cancer is an important goal in need of further advancement.

### 3. SUMMARY

The disclosure provides methods of safely administering STING agonists to patients, particularly in combination with immune checkpoint inhibitors, such as inhibitors of CTLA4, PD-1, and/or PD-L1, particularly antibody inhibitors of these proteins.

In one aspect, the disclosure provides a method of treating a cancer in a patient, comprising conjointly administering a CTLA4 antagonist/inhibitor (e.g., an anti-CTLA4 antibody) and a STING agonist to the patient, wherein the CTLA4 inhibitor is administered intratumorally to the patient. The STING agonist can be administered intratumorally, orally or systemically (e.g., intravenously, intramuscularly, or subcutaneously) to the patient.

In particular embodiments, the CTLA4 inhibitor and the STING agonist are administered intratumorally to the patient. In some such embodiments, the CTLA4 inhibitor and the STING agonist can be administered in a single pharmaceutical composition or can be administered separately, including sequentially, such as first administering the CTLA4 inhibitor and then the STING agonist or vice versa.

In other embodiments, the methods described herein of conjointly administering a CTLA4 inhibitor and a STING agonist further comprise administering, e.g., conjointly, an antagonist/inhibitor of PD-L1 (e.g., an anti-PD-L1 antibody) or an antagonist/inhibitor of PD-1 (e.g., an anti-PD-1 antibody) to the patient. In some such embodiments, the PD-1 or PD-L1 inhibitor may be administered systemically (e.g., intravenously, intramuscularly, or subcutaneously) or intratumorally to the patient.

In another aspect, the disclosure provides methods of augmenting the anti-tumor response of a CTLA4 inhibitor administered intratumorally to a cancer patient, comprising conjointly administering a STING agonist and the CTLA4 inhibitor to the patient. The STING agonist can be administered intratumorally, orally or systemically (e.g., intravenously, intramuscularly, or subcutaneously) to the patient.

In another aspect, the disclosure provides a pharmaceutical composition for intratumoral injection, comprising a CTLA4 inhibitor, a STING agonist, and a pharmaceutically acceptable carrier. In such embodiments, the pharmaceutical composition is suitable for intratumoral injection, meaning that the composition includes one or more pharmaceutically acceptable carriers and/or doses of STING agonist and CTLA4 inhibitor appropriate for intratumoral injection.

In other embodiments, the present disclosure provides a kit for treating a disease or disorder, including cancer, the kit comprising a CTLA4 inhibitor (e.g., an anti-CTLA4 antibody) and a STING agonist. In certain embodiments, the kit provides the CTLA4 inhibitor formulated for intratumoral administration and the STING agonist formulated for intratumoral, oral or systemic (e.g., intravenous, intramuscular, or subcutaneous) administration. In certain embodiments, the kit further comprises a PD-L1 inhibitor (e.g., an anti-PD-L1 antibody) or a PD-1 inhibitor (e.g., an anti-PD-1 antibody). In some of such embodiments, the PD-L1 inhibitor or PD-1 inhibitor are formulated for intratumoral or systemic (e.g., intravenous, intramuscular, or subcutaneous) administration.

In particular embodiments, the disclosure provides methods of treating a cancer patient comprising intratumorally administering a CTLA4 inhibitor (e.g., an anti-CTLA4 antibody) conjointly with a compound (“Compound A”) having the following structure, or a pharmaceutically acceptable salt thereof:

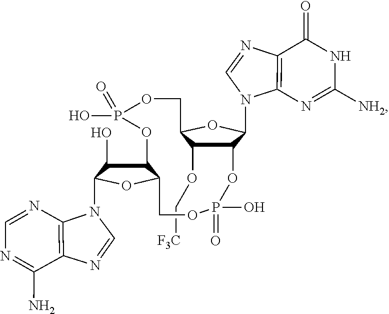

wherein compound A is administered intratumorally or systemically (e.g., intravenously, intramuscularly, or subcutaneously) to the patient. Compound A is a cyclic dinucleotide that is capable of activating STING and was described in U.S. Published Application No. 2018/0230177, which is incorporated herein by reference. Various salt forms of Compound A can be administered to a cancer patient. For instance, in one embodiment, a therapeutically effective amount of a sodium salt of Compound A is administered to the cancer patient. It will be understood that any reference to Compound A in the disclosure also includes pharmaceutically acceptable salts thereof.

In another aspect, the disclosure provides methods of treating cancer, comprising conjointly administering a STING agonist to the cancer patient, wherein the dosing regimen comprises administration of a priming dose of the STING agonist at the onset of the therapy, followed by administration of maintenance doses of the STING agonist. The STING agonist can be administered intratumorally, orally or systemically. The STING agonist can be administered by itself or conjointly with one or more anti-cancer agents. For instance, the STING agonist can be administered conjointly with a CTLA4 inhibitor, PD-1 inhibitor or PD-L1 inhibitor, or a combination thereof. In particular embodiments, the CTLA4 inhibitor, PD-1 inhibitor or PD-L1 inhibitor can be administered intratumorally or systemically. In some such embodiments, the STING agonist and CTLA4 inhibitor can be administered intratumorally.

### 4. BRIEF DESCRIPTION OF THE FIGURES

FIG. 1, panels A and B show the effect of intratumoral administration of Compound A and anti-CTLA4 antibody. Groups of C57BL6 mice (n=5) bearing B16F10 tumors were treated as indicated on day 6, 10, and 14 after tumor implantation. FIG. 1, panel A shows tumor growth over time, and FIG. 1, panel B shows mice survival over time. Data are shown as mean±SEM.

FIG. 2, panels A and B show the effect of a triple combination of Compound A (I.T.), PD-L1 antibody (I.P.), and anti-CTLA4 antibody (I.P.). Groups of C57BL6 mice (n=5) bearing B16F10 tumors were treated as indicated on day 6, 10, and 14 after tumor implantation. FIG. 2, panel A shows tumor growth over time, and FIG. 2, panel B shows mice survival over time. Data are shown as mean±SEM.

FIG. 3, panels A and B show the anti-tumor efficacy of DMXAA (which is 5,6-dimethylxanthenone-4-acetic acid, a known STING agonist) and anti-CTLA4 antibody. Groups of C57BL6/J mice (n=5) were implanted subcutaneously with B16F10 melanoma cells into the right flank on day 0. On day 6, 9, 12, and 15, mice were treated intratumorally with anti-CTLA4 antibody or DMXAA, or the combination of both anti-CTLA4 antibody and DMXAA. FIG. 3, panel A shows tumor growth over time, and FIG. 3, panel B shows mice survival over time. Data are shown as mean±SEM.

### 5. DETAILED DESCRIPTION

5.1. Intratumoral Administration of CTLA4 Inhibitors in Combination with STING Agonists

The disclosure provides methods of treating a disease or disorder, particularly cancer, in a patient in need thereof, comprising administering in combination (e.g., conjointly) a CTLA4 inhibitor (e.g., an anti-CTLA4 antibody) and a STING agonist to the patient, wherein the CTLA4 inhibitor is administered intratumorally. In certain embodiments, the CTLA4 inhibitor and the STING agonist are administered conjointly to the patient. Conjoint administration refers to administration of one therapeutic agent (e.g., a CTLA4 inhibitor) when another therapeutic agent (e.g., a STING agonist), having been previously administered to the patient, is still efficacious in the body of the patient. Conjoint administration contemplates that the CTLA4 inhibitor can be administered simultaneously, prior to, or after administration of the STING agonist.

In some embodiments, the CTLA4 inhibitor and the STING agonist can both be administered intratumorally to a patient. In these embodiments, the STING agonist and the CTLA4 inhibitor can be administered together in the same pharmaceutical composition or in separate pharmaceutical compositions. In other embodiments, the CTLA4 inhibitor can be administered intratumorally to the patient and the STING agonist can be administered systemically (e.g., intravenously, intramuscularly, or subcutaneously) to the patient. In still other embodiments, the CTLA4 inhibitor can be administered intratumorally to the patient and the STING agonist can be administered orally to the patient.

In embodiments where the CTLA4 inhibitor and the STING agonist are administered in separate compositions, the two compositions can be administered concomitantly or sequentially. In particular embodiments where the CTLA4 inhibitor and the STING agonist are administered sequentially, the STING agonist can be administered prior to the administration of the CTLA4 inhibitor. Alternatively, the STING agonist can be administered after administration of the CTLA4 inhibitor.

In some embodiments, the CTLA4 inhibitor and the STING agonist can be administered in combination, e.g., conjointly, without any additional therapeutic agents. Surprisingly, for some tumors, such as those exemplified herein, the combination of CTLA4 inhibitor and the STING agonist provides sufficient tumor inhibition such that additional chemotherapeutic agents or immunotherapeutic agents may not provide additional tumor inhibition.

Nonetheless, in some embodiments, additional clinical benefit may be achieved by administration with other therapeutic agents. Accordingly, in some embodiments, the CTLA4 inhibitor and the STING agonist can be administered in combination, e.g., conjointly, with other therapeutic agents. For instance, the CTLA4 inhibitor and the STING agonist can be administered conjointly with one additional immune checkpoint inhibitor. In particular embodiments, the CTLA4 inhibitor and the STING agonist can be administered as part of a triple combination with a PD-1 inhibitor or a PD-L1 inhibitor (e.g., an anti-PD-1 antibody or anti-PD-L1 antibody).

In one embodiment, the CTLA4 inhibitor and the STING agonist can be administered to a cancer in combination, e.g., conjointly, with a PD-1 or PD-L1 inhibitor, such as those described herein. In such cases, the PD-1 or PD-L1 inhibitor can be administered simultaneously with, prior to or after administration of the CTLA4 inhibitor and/or the STING agonist. In some embodiments, the PD-1 or PD-L1 inhibitor can be administered intratumorally. In other embodiments, the PD-1 or PD-L1 inhibitor can be administered systemically, such as intravenously, subcutaneously, or intramuscularly. In certain embodiments, both the CTLA4 inhibitor and STING agonist are administered intratumorally to the cancer patient, and the PD-L1 inhibitor or PD-1 inhibitor is administered systemically. In other embodiments, the CTLA4 inhibitor is administered intratumorally to the cancer patient, and both the STING agonist and the PD-L1 inhibitor or PD-1 inhibitor are administered systemically. In certain embodiments, both the CTLA4 inhibitor and PD-L1 inhibitor or PD-1 inhibitor are administered intratumorally to the cancer patient, and the STING agonist is administered systemically. In some embodiments, both the CTLA4 inhibitor and PD-L1 inhibitor or PD-1 inhibitor are administered intratumorally to the cancer patient, and the STING agonist is administered orally. In other embodiments, the CTLA4 inhibitor, the STING agonist, and the PD-L1 inhibitor or PD-1 inhibitor are all administered intratumorally to the cancer patient. In yet other embodiments, the CTLA4 inhibitor is administered intratumoral to the cancer patient, the STING agonist is administered orally to the patient, and the PD-L1 inhibitor or PD-1 inhibitor is administered systemically to the cancer patient.

In some embodiments, the CTLA4 inhibitor inhibits the interaction between CTLA4 on T cells and CD80 (B7.1) or CD86 (B7.2) on an antigen presenting cell such as a dendritic cell or a macrophage in the tumor microenvironment.

Intratumoral administration of a CTLA4 inhibitor (e.g., an anti-CTLA4 antibody) mitigates the safety problems associated with systemic administration of the CTLA4 inhibitor, albeit potentially at the cost of reduced efficacy. As disclosed herein, the efficacy associated with intratumoral administration of a CTLA4 inhibitor can be significantly enhanced when the CTLA4 inhibitor is administered conjointly with a STING agonist. The STING agonist can be administered intratumorally, systemically or orally. Administration of the STING agonist, as disclosed herein, overcomes the prior art safety and efficacy problems. Specifically, when a CTLA4 inhibitor and a STING agonist are administered conjointly, the STING agonist synergizes with the CTLA4 inhibitor, producing an effect significantly greater than the sum of their parts (i.e., more than an additive effect). Accordingly, the dose of the STING agonist and/or the CTLA4 inhibitor required to treat a tumor, when used in combination, is lower than the doses required when the STING agonist and the CTLA4 inhibitor are administered individually. As demonstrated herein, the enhanced tumor response can be shown by shrinkage of the tumor or by increased survival times

Surprisingly, as shown in Example 2 herein, the ability of a particular STING agonist (Compound A) to potentiate the anti-tumor effect of an anti-CTLA4 antibody is significantly greater when the anti-CTLA4 antibody is administered intratumorally than when the anti-CTLA4 antibody is administered systemically. Specifically, when administered in combination with an intratumoral dose of Compound A, the low dose (50 μg) intratumoral administration of the anti-CTLA4 antibody to diseased mice provided significant benefits in terms of tumor size and overall survival when compared to the higher dose (200 μg) of the anti-CTLA4 antibody administered systemically. In fact, even when the intratumoral dose of the anti-CTLA4 antibody was decreased 5-fold (to 10 μg), the anti-tumor effect was similar to that of 200 μg of the anti-CTLA4 antibody administered systemically.

Therefore, the present disclosure shows that the anti-tumor effect of a CTLA4 inhibitor administered intratumorally can be significantly enhanced by conjoint intratumoral administration of a STING agonist. Accordingly, in one aspect, the disclosure provides methods of augmenting the anti-tumor response of a CTLA4 inhibitor administered intratumorally to a cancer patient, comprising intratumorally and conjointly administering a STING agonist and the CTLA4 inhibitor to the patient. As demonstrated herein, the enhanced tumor response can be shown by shrinkage of the tumor or by increased survival times.

In one aspect, the disclosure provides methods of treating or preventing metastasis in a human cancer patient comprising conjointly administering to a cancer patient an intratumoral dose of a CTLA4 inhibitor with a therapeutically effective amount of a STING agonist. In certain embodiments, the STING agonist is administered intratumorally, either in the same pharmaceutical composition as the CTLA4 inhibitor or in a different composition than the CTLA4 inhibitor. In other embodiments the STING agonist is administered systemically (e.g., subcutaneously, intramuscularly, or intravenously). In still other embodiments, the STING agonist is administered orally. In certain embodiments, the CTLA4 inhibitor and the STING agonist are administered conjointly with a PD-1 inhibitor or a PD-L1 inhibitor.

In some embodiments of the disclosure, the STING agonist can be combined with the intratumoral dose of the CTLA4 inhibitor to treat cancers that are resistant or refractory to immune checkpoint therapy. For instance, the combination therapy can be used to treat primary or metastasizing tumors that are resistant to immune checkpoint therapy. In some such embodiments, the CTLA4 inhibitor and the STING agonist are administered conjointly with a PD-1 inhibitor or a PD-L1 inhibitor.

In one embodiment, the STING agonist is administered to a human cancer patient already receiving immune checkpoint inhibition therapy, such as for whom the cancer has stabilized. In particular embodiments, the cancer patient has undergone at least 1 or 2 cycles of immune checkpoint inhibitor therapy prior to administration of the STING agonist and the intratumoral dose of the CTLA4 inhibitor. For instance, the cancer patient may have undergone 2, 3, 4, 5, 6, 7, or 8 cycles of immune checkpoint inhibition therapy prior to administration of the STING agonist and the intratumoral dose of the CTLA4 inhibitor. In certain of these embodiments, the cancer patient continues to receive immune checkpoint inhibition therapy with successive cycles of the STING agonist is administered.

In certain embodiments, the STING agonist administered in combination, e.g., conjointly, with the CTLA4 inhibitor is a cyclic dinucleotide (CDN) compound. For instance, the STING agonist can be a 2′3′-CDN, such as 2′3′-cGAMP or Compound A, depicted above. In other embodiments, the STING agonist is a 3′3′-CDN, a 2′2′-CDN, or a 3′2′-CDN. In some embodiments, the STING agonist is a benzophenone analog. In further embodiments, the STING agonist is a dimeric amidobenzimidazole. Examples of STING agonists that can be used in accordance with the disclosure include ADU-S100 (MIW815), BMS-986301, CRD5500, CMA (10-carboxymethyl-9-acridanone), diABZI STING agonist-1 (e.g., CAS No.: 2138299-34-8), DMXAA (ASA404/vadimezan), E7766, GSK-532, GSK-3745417, MK-1454, MK-2118, SB-11285, SRCB-0074, TAK-676, TTI-10001, SR-717 and MSA-2.

In one embodiment, the CDN administered in accordance with the disclosure is the following compound (“Compound A”), or a pharmaceutically acceptable salt thereof:

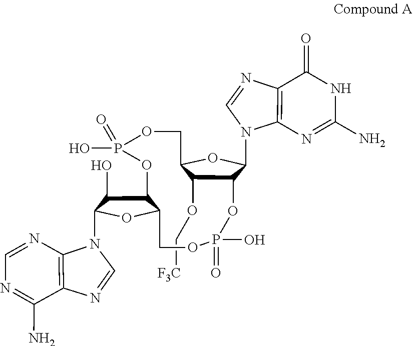

Compound A can act both locally and systemically to exert a powerful anti-tumor effect. Compound A, when administered at particular dosages to a cancer patient in need thereof, is capable of substantially reducing or preventing the spreading of metastasis. The ability of Compound A to reduce or prevent the onset and/or progression of metastasis can be potentiated when administered conjointly with an intratumoral dose of a CTLA4 inhibitor, in accordance with the disclosure. Additionally, it has been discovered that Compound A exerts a powerful abscopal effect when administered conjointly with an intratumoral dose of a CTLA4 inhibitor, in accordance with the present disclosure.

In some embodiments where Compound A serves as the STING agonist to be administered conjointly with the intratumoral dose of the CTLA4 inhibitor, Compound A can be administered over multiple cycles. For instance, in one embodiment, the first cycle comprises administering Compound A on days 1, 8, and 15 of a four-week period, and subsequent cycles comprise administering Compound A on days 1 and 15 (i.e., biweekly) of a four-week period. Compound A can be administered intratumorally or systemically, including subcutaneously, intramuscularly, or intravenously. In some embodiments, on days of the cycle designated for administration, Compound A can be administered at a dosage in the range of 50 μg to 6,500 μg. In some embodiments, on days of the cycle designated for administration, Compound A can be administered at a dosage in the range of 100 μg to 3,000 μg. In some embodiments, on days of the cycle designated for administration, Compound A can be administered at a dosage in the range of 100 μg to 1,200 μg.

In one embodiment, the CDN administered in accordance with the disclosure is the following compound (“Compound B”), or a pharmaceutically acceptable salt thereof:

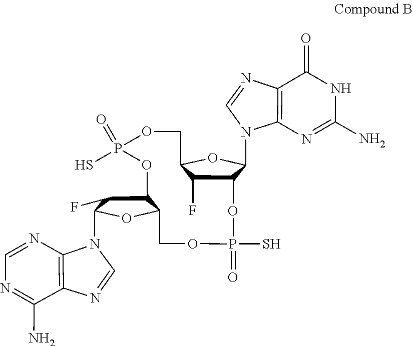

In another embodiment, the CDN administered in accordance with the disclosure is the following compound (“Compound C”), or a pharmaceutically acceptable salt thereof:

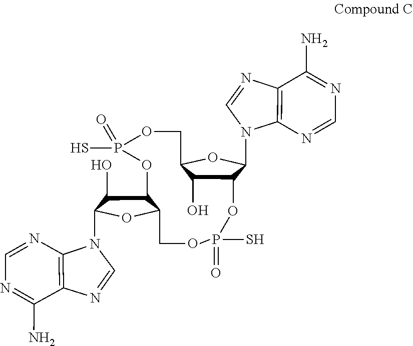

In another embodiment, the STING agonist administered in accordance with the disclosure is a compound as disclosed in WO 2019/165032, which is herein incorporated by reference. Such STING agonists can be administered orally, systemically, or intratumorally to the patient. An example of one such STING agonist that can be administered in accordance with the disclosure is SR-717 (“Compound D”), or a pharmaceutically acceptable salt thereof, which has the following structure:

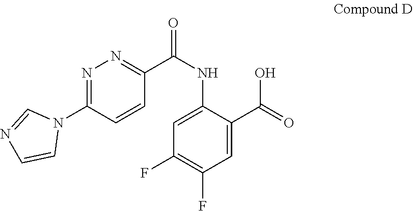 [SMILES: O=C(Nc1cc(F)c(F)cc1C(=O)O)c1ccc(-n2ccnc2)nn1]

In another embodiment, the STING agonist administered in accordance with the disclosure is MSA-2 (“Compound E”), or a pharmaceutically acceptable salt thereof, which has the following structure:

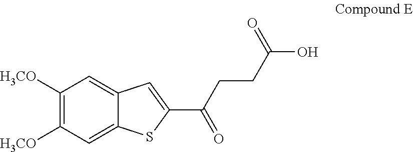 [SMILES: COc1cc2cc(C(=O)CCC(=O)O)sc2cc1OC]

MSA-2 can be administered orally, systemically, or intratumorally to the patient.

Additional examples of CDNs that can be used as STING agonists in the present methods are disclosed in the following publications WO 2014/144666, WO 2014/179335, WO 2014/189806, WO 2015/161762, WO 2016/096174, WO 2017/027646, WO 2017/027645, WO 2017/161349, WO 2018/118664, WO 2018/118665, WO 2018/208667, WO2019/165032, and WO 2019/046511 the contents of each of which are incorporated by reference herein.

In other embodiments, the STING agonist to be administered in accordance with the disclosure can be conjugated to antibodies or antigen-binding fragments, hence producing antibody-drug conjugates (ADCs).

In one embodiment, the ADC to be administered in accordance with the disclosure has a structure as described in US 2017/0298139, WO 2017/100305, WO 2018/200812, or WO 2018/140831, the contents of each of which are herein incorporated by reference herein.

In particular embodiments, the ADC to be administered in accordance with the disclosure has the structure of Formula IA:

Ab-[-L-D]ₙ (IA)
wherein:
“D” represents a CDN having the structure of Formula IIa:

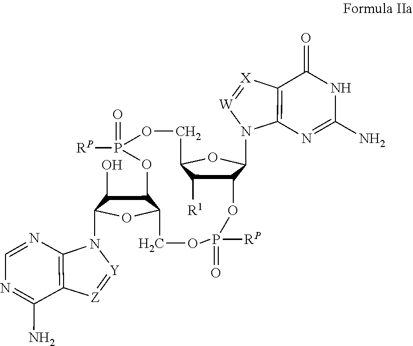

wherein
   W, X, Y, and Z are independently CH or N;
   R¹ is C₂₋₄alkyl substituted with a thiol, amino, or C₁₋₆alkylamino group;
   R^{p} is, independently for each occurrence, hydroxyl, thiol, C₁₋₆alkyl, borano (—BH₃⁻), or —NR′R″, wherein R′ and R″ are, independently for each occurrence, hydrogen or C₁₋₆alkyl optionally substituted with one or more groups selected from halogen, thiol, hydroxyl, carboxyl, C₁₋₆alkoxy, C₁₋₆hydroxyalkoxy, —OC(O)C₁₋₆alkyl, —N(H)C(O)C₁₋₆alkyl, —N(C₁₋₃alkyl)C(O)C₁₋₆alkyl, amino, C₁₋₆alkylamino, di(C₁₋₆alkyl)amino, oxo, and azido; or R′ and R″ on the same nitrogen together form a C₃₋₅heterocyclic ring;
or a pharmaceutically acceptable salt thereof;
“Ab” represents an antibody or binding fragment thereof which binds a target antigen;
“L” represents, independently for each occurrence, a linker linking one or more occurrences of D to Ab;
“n” represents the number of occurrences of D linked to Ab via the linker (L);

wherein the CDN (D) is covalently bound to linker (L) at the thiol, amino, or C₁₋₆alkylamino group at the R¹ position of the CDN.

In some embodiments wherein the STING agonist is administered as part of an ADC of Formula IA, the CDN of the ADC has he structure of Formula IIb:

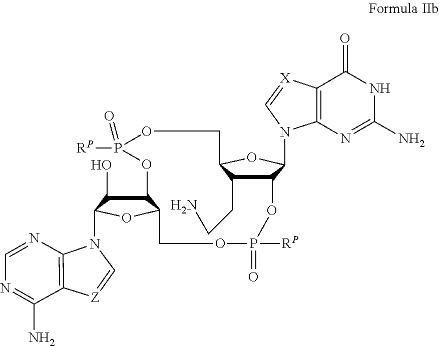

or a pharmaceutically acceptable salt thereof.

In some embodiments wherein the STING agonist is administered as part of an ADC of Formula IA, the CDN of the ADC has he structure of Formula IIc:

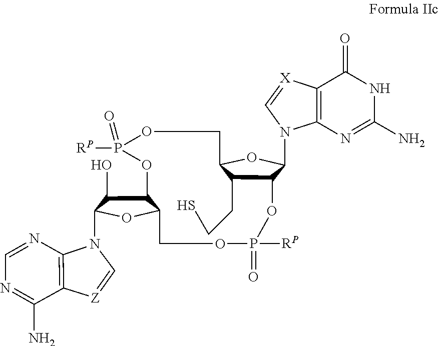

or a pharmaceutically acceptable salt thereof.

In some embodiments wherein the STING agonist is administered as part of an ADC of Formula IA, the ADC has the structure of Formula III:

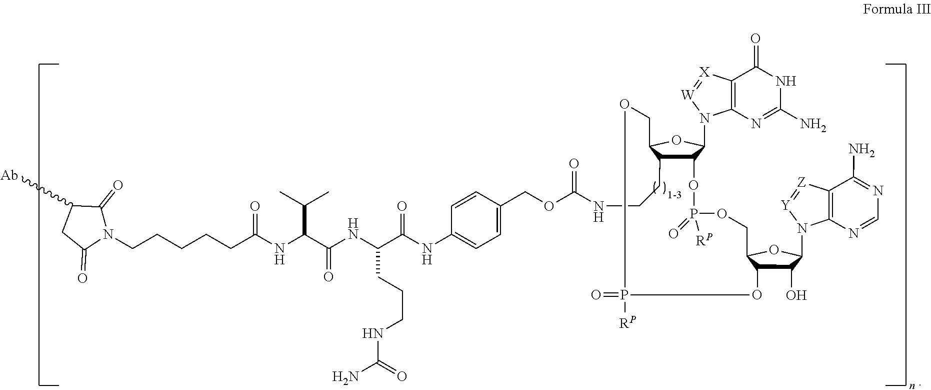

In some embodiments wherein the STING agonist is administered as part of an ADC of Formula IA, the ADC has the structure of Formula IV:

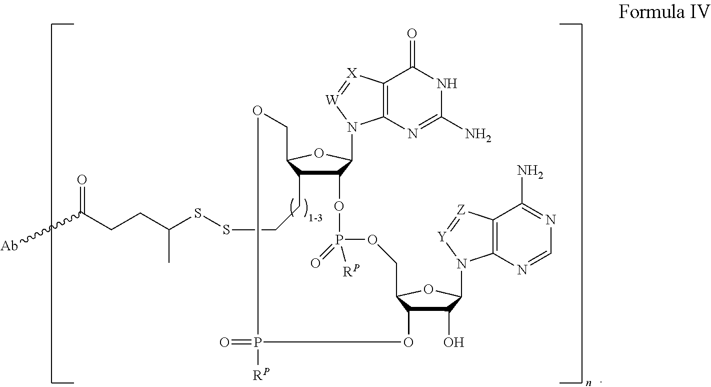

In some embodiments wherein the STING agonist is administered as part of an ADC of Formula IA, the ADC (“Compound F”) has the following structure:

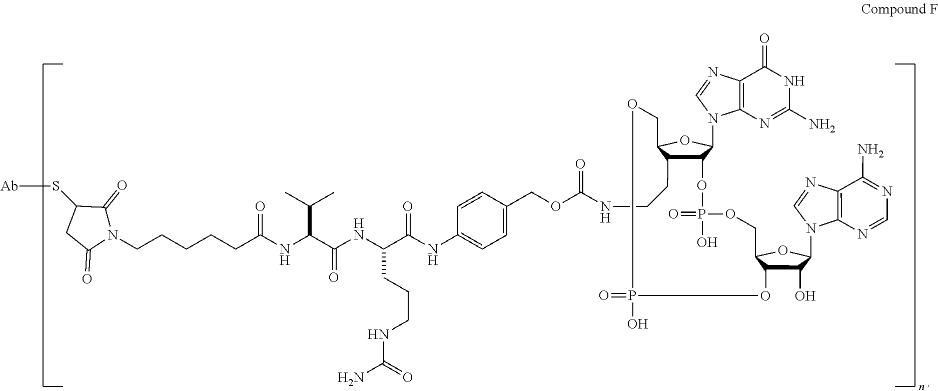

In some embodiments wherein the STING agonist is administered as part of an ADC of Formula IA, the ADC (“Compound G”) has the following structure:

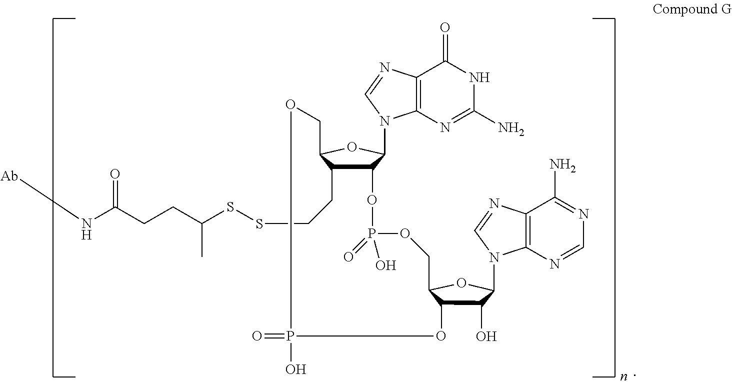

Examples of CTLA4 inhibitors that can be used in accordance with the present disclosure include, but are not limited to, ipilimumab (Yervoy®) and tremelimumab (ticilimumab), CBT-509, CS1002, BMS-986249, AGEN1181, AGEN1194, AGN2041, BA3071, ATOR-1015, ATOR-1144, ADV-1604 and BCD-145. In particular embodiments, the CTLA4 inhibitor is an anti-CTLA4 antibody selected from ipilimumab (Yervoy®) and tremelimumab.

In some embodiments where a PD-1 inhibitor is administered in combination with the CTLA4 inhibitor and the STING agonist, the PD-1 inhibitor can be, but is not limited to, pembrolizumab (Keytruda®), nivolumab (Opdivo®), cemiplimab (Libtayo®), AMP-224, AMP-514, or PDR001. The PD-1 inhibitor can generally be administered systemically or intratumorally.

In some embodiments where a PD-L1 inhibitor is administered in combination with the CTLA4 inhibitor and the STING agonist, the PD-L1 inhibitor can be, but is not limited to, atezolizumab (Tecentriq®), avelumab (Bavencio®), urvalumab (Imfinzi®), BMS-936559, or CK-301. The PD-L1 inhibitor can generally be administered systemically or intratumorally.

In particular embodiments, the anti-CTLA4 antibody ipilimumab is administered intratumorally and conjointly with Compound A, which may be administered intratumorally or systemically. In such embodiments, the combination of ipilumumab and Compound A may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound A may be conjointly administered with a PD-L1 inhibitor selected from atezolizumab (Tecentriq®), avelumab (Bavencio®), urvalumab (Imfinzi®), BMS-936559, or CK-301.

In particular embodiments, the anti-CTLA4 antibody ipilimumab is administered intratumorally and conjointly with Compound A, which may be administered intratumorally or systemically. In such embodiments, the combination of ipilumumab and Compound A may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound A may be conjointly administered with a PD-L1 inhibitor selected from atezolizumab (Tecentriq®), avelumab (Bavencio®), urvalumab (Imfinzi®), BMS-936559, or CK-301.

In particular embodiments, the anti-CTLA4 antibody ipilimumab is administered intratumorally and conjointly with Compound B, which may be administered intratumorally or systemically. In such embodiments, the combination of ipilumumab and Compound B may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound B may be conjointly administered with a PD-L1 inhibitor selected from atezolizumab (Tecentriq®), avelumab (Bavencio®), urvalumab (Imfinzi®), BMS-936559, or CK-301.

In particular embodiments, the anti-CTLA4 antibody ipilimumab is administered intratumorally and conjointly with Compound C, which may be administered intratumorally or systemically. In such embodiments, the combination of ipilumumab and Compound C may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound C may be conjointly administered with a PD-L1 inhibitor selected from atezolizumab (Tecentriq®), avelumab (Bavencio®), urvalumab (Imfinzi®), BMS-936559, or CK-301.

In particular embodiments, the anti-CTLA4 antibody ipilimumab is administered intratumorally and conjointly with Compound D, which may be administered intratumorally or systemically. In such embodiments, the combination of ipilumumab and Compound D may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound D may be conjointly administered with a PD-L1 inhibitor selected from atezolizumab (Tecentriq®), avelumab (Bavencio®), urvalumab (Imfinzi®), BMS-936559, or CK-301.

In particular embodiments, the anti-CTLA4 antibody ipilimumab is administered intratumorally and conjointly with Compound E, which may be administered intratumorally or systemically. In such embodiments, the combination of ipilumumab and Compound E may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound E may be conjointly administered with a PD-L1 inhibitor selected from atezolizumab (Tecentriq®), avelumab (Bavencio®), urvalumab (Imfinzi®), BMS-936559, or CK-301.

In particular embodiments, the anti-CTLA4 antibody ipilimumab is administered intratumorally and conjointly with Compound F, which may be administered intratumorally or systemically. In such embodiments, the combination of ipilumumab and Compound F may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound F may be conjointly administered with a PD-L1 inhibitor selected from atezolizumab (Tecentriq®), avelumab (Bavencio®), urvalumab (Imfinzi®), BMS-936559, or CK-301.

In particular embodiments, the anti-CTLA4 antibody ipilimumab is administered intratumorally and conjointly with Compound G, which may be administered intratumorally or systemically. In such embodiments, the combination of ipilumumab and Compound G may be conjointly administered with a PD-1 inhibitor selected from pembrolizumab (Keytruda®), nivolumab (Opdivo®), cemiplimab (Libtayo®), AMP-224, AMP-514, and PDR001. Alternatively, the combination of ipilumumab and Compound G may be conjointly administered with a PD-L1 inhibitor selected from atezolizumab (Tecentriq®), avelumab (Bavencio®), urvalumab (Imfinzi®), BMS-936559, or CK-301.

5.2. Further Methods of Treatment

The combination therapies disclosed herein can be used to treat a disease or disorder, particularly cancer. In accordance with the disclosure, the combination therapies can be used to treat both primary tumors and metastasizing tumors. In some embodiments, the CTLA4 inhibitor, STING agonist and optionally one or more additional anti-cancer agents (e.g., a PD-1 or PD-L1 inhibitor) can be administered at dosage levels or under a particular dosing regimen as disclosed herein that results in shrinking or eradicating primary tumors and developing metastases stemming from the primary tumors.

Accordingly, in one aspect, the disclosure provides methods of treating cancer in a subject comprising conjointly administering a CTLA4 inhibitor, a STING agonist and optionally one or more additional anti-cancer agents (e.g., a PD-1 or PD-L1 inhibitor), wherein the CTLA4 inhibitor is administered intratumorally. The STING agonist and additional anti-cancer agents may be administered intratumorally, systemically or orally. The CTLA4 inhibitor, a STING agonist and optionally one or more additional anti-cancer agents can be administered together in a single pharmaceutical composition. Alternatively, the CTLA4 inhibitor, a STING agonist and optionally one or more additional anti-cancer agents can be administered in separate pharmaceutical compositions. In some embodiments, the pharmaceutical compositions are administered to mammals in need thereof. In particular embodiments, the pharmaceutical compositions are administered to a human patient in need thereof.

In some embodiments, both the CTLA4 inhibitor and the STING agonist are administered intratumorally into the primary tumor of the patient. It has been found that when particular STING agonists (e.g., Compound A) are administered intratumorally into the primary tumor, tumor growth is suppressed not only at the site of the primary tumor, but also at the site of distant tumors. Therefore, such STING agonists display an abscopal effect. Moreover, the STING agonist potentiates the checkpoint modulation of CTLA4 by augmenting T cell priming and inflammation in the tumor microenvironment, at both the site of injection and at distal legions. Accordingly, the abscopal potential of CTLA4 inhibition is enhanced through co-administration with the STING agonist.

Accordingly, the disclosure provides methods of treating both primary and distant tumors (including accessible and inaccessible cancers) by administering the combination therapies disclosed herein.

In some embodiments, the STING agonist is administered systemically to the patient. For instance, the STING agonist can be administered intravenously, intramuscularly, or subcutaneously to a cancer patient.

In certain embodiments, the STING agonist can be administered orally. In some such embodiments, the oral STING agonist is SR-717 or MSA-2.

The present disclosure also provides a method of treating a patient, who is concurrently being treated with intratumoral doses of a CTLA4 inhibitor (e.g., an anti-CTLA4 antibody) as described herein, comprising administering to the patient a STING agonist as described herein. In certain embodiments, the STING agonist is administered intratumorally. In other embodiments, the STING agonist is administered systemically (e.g., intravenously, intramuscularly, or subcutaneously). In further embodiments, the STING agonist is administered orally. In some embodiments, the method further comprises administering a PD-L1 inhibitor (e.g., an anti-PD-L1 antibody) or a PD-1 inhibitor (e.g., an anti-PD-1 antibody) as described herein to the patient. In certain of these embodiments, the patient is suffering from a cancer, such as those described herein. In some embodiments, the method of treating the patient treats the patient for the cancer.

The present disclosure also provides a method of treating a patient, who is concurrently being treated with a STING agonist as described herein, comprising intratumorally administering a CTLA4 inhibitor (e.g., an anti-CTLA4 antibody) as described herein to the patient. In some embodiments, the method further comprises administering a PD-L1 inhibitor (e.g., an anti-PD-L1 antibody) or a PD-1 inhibitor (e.g., an anti-PD-1 antibody) as described herein to the patient. In certain of these embodiments, the patient is suffering from a cancer, such as those described herein. In some embodiments, the method of treating the patient treats the patient for the cancer.

In particular embodiments, the combination therapies of the disclosure can be used to treat cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney, or liver. Further cancers treatable by the combination therapies of the disclosure include rectal cancer; cancer of the anal region; carcinomas of the fallopian tubes, endometrium, cervix, vagina, vulva, renal pelvis, and renal cell; sarcoma of soft tissue; myxoma; rhabdomyoma; fibroma; lipoma; teratoma; cholangiocarcinoma; hepatoblastoma; angiosarcoma; hemagioma; hepatoma; fibrosarcoma; chondrosarcoma; myeloma; chronic or acute leukemia; lymphocytic lymphomas; primary CNS lymphoma; neoplasms of the CNS; spinal axis tumors; squamous cell carcinomas; synovial sarcoma; malignant pleural mesotheliomas; brain stem glioma; pituitary adenoma; bronchial adenoma; chondromatous hanlartoma; inesothelioma; Hodgkin's Disease; or a combination of one or more of the foregoing cancers.

In particular embodiments, the combination therapies of the disclosure can be used to treat a cancer that is refractory or unresponsive to immune checkpoint inhibitory therapy. Such cancers may include but are not limited to prostate cancer, pancreatic cancer, lymphoma, head and neck cancer, kidney cancer, melanoma, colon cancer, breast cancer, and lung cancer. In certain embodiments, the cancer is selected from prostate cancer, pancreatic cancer, lymphoma, head and neck cancer, and kidney cancer. In some embodiments, the cancer is selected from melanoma, colon cancer, breast cancer, and lung cancer.

5.3. Pharmaceutical Compositions, Kits, and Combination Therapies

The disclosure further provides for a pharmaceutical composition comprising a CTLA4 inhibitor, a STING agonist, and a pharmaceutically acceptable carrier. In certain embodiments, the pharmaceutical composition is an injectable pharmaceutical composition, e.g., for intratumoral injection. In some embodiments, the pharmaceutical acceptable carrier may include physiological saline or phosphate buffered saline (PBS). A particular advantage provided by the disclosure is that the STING agonist and the CTLA4 inhibitor can be administered intratumorally in a single composition. Administration of a single composition reduces the number of injections required and reduces incidence of side effects associated with administration of multiple doses of the individual therapeutic agents. Moreover, because of the synergy observed when the CTLA4 inhibitor is administered together with the STING agonist, the dose of either of the agents to achieve efficacy is less than the dose to achieve efficacy when either of the agents is administered as a monotherapy. Accordingly, incidence of side effects such as irritation is further reduced by this synergy.

In other embodiments, the present disclosure provides a kit for treating a disease or disorder, including cancer, the kit comprising a CTLA4 inhibitor (e.g., an anti-CTLA4 antibody) and a STING agonist. In certain embodiments, the kit provides the CTLA4 inhibitor formulated for intratumoral administration and the STING agonist formulated for intratumoral, oral or systemic (e.g., intravenous, intramuscular, or subcutaneous) administration. In some embodiments, both the CTLA4 inhibitor and STING agonist are formulated for intratumoral administration. In other embodiments, the CTLA4 inhibitor is formulated for intratumoral administration, and the STING agonist is formulated for systemic administration. In yet other embodiments, the CTLA4 inhibitor is formulated for intratumoral administration, and the STING agonist is formulated for oral administration.

In certain embodiments, the kit further comprises a PD-L1 inhibitor (e.g., an anti-PD-L1 antibody) or a PD-1 inhibitor (e.g., an anti-PD-1 antibody). In some of such embodiments, the PD-L1 inhibitor or PD-1 inhibitor are formulated for intratumoral or systemic (e.g., intravenous, intramuscular, or subcutaneous) administration. In certain embodiments, both the CTLA4 inhibitor and STING agonist are formulated for intratumoral administration, and the PD-L1 inhibitor or PD-1 inhibitor is formulated for systemic administration. In other embodiments, the CTLA4 inhibitor is formulated for intratumoral administration, and both the STING agonist and the PD-L1 inhibitor or PD-1 inhibitor are formulated for systemic administration. In certain embodiments, both the CTLA4 inhibitor and PD-L1 inhibitor or PD-1 inhibitor are formulated for intratumoral administration, and the STING agonist is formulated for systemic administration. In some embodiments, both the CTLA4 inhibitor and PD-L1 inhibitor or PD-1 inhibitor are formulated for intratumoral administration, and the STING agonist is formulated for oral administration. In other embodiments, the CTLA4 inhibitor, the STING agonist, and the PD-L1 inhibitor or PD-1 inhibitor are all formulated for intratumoral administration. In yet other embodiments, the CTLA4 inhibitor is formulated for intratumoral administration, the STING agonist is formulated for oral administration, and the PD-L1 inhibitor or PD-1 inhibitor is formulated systemic administration.

The present disclosure also provides a combination therapy, for example for treating a cancer as described herein, wherein the combination therapy comprises an intratumoral administration regimen of a CTLA4 inhibitor (e.g., an anti-CTLA4 antibody) and a regimen of a STING agonist as described herein. The STING agonist regimen may be an intratumoral, oral or systemic (e.g., intravenous, intramuscular, or subcutaneous) administration regimen. In some embodiments, the combination therapy further comprises a regimen of a PD-L1 inhibitor (e.g., an anti-PD-L1 antibody) or a PD-1 inhibitor (e.g., an anti-PD-1 antibody). The PD-L1 inhibitor or PD-1 inhibitor regimen may be an intratumoral or systemic (e.g., intravenous, intramuscular, or subcutaneous) administration regimen.

In certain embodiments, the combination therapy comprises an intratumoral administration regimen of a CTLA4 inhibitor, an intratumoral administration regimen of a STING agonist, and an intratumoral administration regimen of a PD-L1 inhibitor or PD-1 inhibitor. In other embodiments, the combination therapy comprises an intratumoral administration regimen of a CTLA4 inhibitor, a systemic administration regimen of a STING agonist, and an intratumoral administration regimen of a PD-L1 inhibitor or PD-1 inhibitor. In other embodiments, the combination therapy comprises an intratumoral administration regimen of a CTLA4 inhibitor, an intratumoral administration regimen of a STING agonist, and a systemic administration regimen of a PD-L1 inhibitor or PD-1 inhibitor. In other embodiments, the combination therapy comprises an intratumoral administration regimen of a CTLA4 inhibitor, a systemic administration regimen of a STING agonist, and a systemic administration regimen of a PD-L1 inhibitor or PD-1 inhibitor. In other embodiments, the combination therapy comprises an intratumoral administration regimen of a CTLA4 inhibitor, an oral administration regimen of a STING agonist, and a systemic administration regimen of a PD-L1 inhibitor or PD-1 inhibitor. And in yet other embodiments, the combination therapy comprises an intratumoral administration regimen of a CTLA4 inhibitor, an oral administration regimen of a STING agonist, and an intratumoral administration regimen of a PD-L1 inhibitor or PD-1 inhibitor.

5.4. Dosing Regimens

A particular advantage associated with intratumoral administration of a CTLA4 inhibitor is that it can be delivered at doses less than the systemic route of administration. However, intratumoral administration of a CTLA4 inhibitor may provide limited anti-cancer efficacy. As disclosed herein, the anti-tumor effect of a low dose of a CTLA4 inhibitor administered intratumorally can be markedly enhanced by conjoint administration with a STING agonist. “Low dose” administration of the CTLA4 inhibitor refers to a dose of the CTLA4 inhibitor that is significantly lower than the dose of the CTLA4 inhibitor that is known to have a therapeutic effect when administered systemically. For instance, “low dose” administration of a commercially available CTLA4 inhibitor may refer to a dose of the CTLA4 inhibitor that is significantly lower than the therapeutically effective dose of the CTLA4 inhibitor administered to the patient systemically, e.g., as reflected on the CTLA4 inhibitor's product label. For instance, the intratumoral dose of the CTLA4 inhibitor can be from 2-fold to 50-fold less than the therapeutically effective dose of the CTLA4 inhibitor, e.g., as reflected on the product label. In some embodiments, the intratumoral dose of the CTLA4 inhibitor can be from 3-fold to 50-fold less than the therapeutically effective dose of the CTLA4 inhibitor, e.g., as reflected on the product label. In other embodiments, the intratumoral dose of the CTLA4 inhibitor can be from 4-fold to 10-fold less than the therapeutically effective dose of the CTLA4 inhibitor, e.g., as reflected on the product label.

The particular dose and dosing regimen of the CTLA4 inhibitor administered in combination with the STING agonist will depend on the particular CTLA4 inhibitor and the cancer being treated. In embodiments where the CTLA4 inhibitor is an anti-CS1 antibody, the antibody can be administered every 1-4 weeks. In particular embodiments, the STING agonist can be administered on a weekly, biweekly, triweekly, or monthly basis. In such embodiments, the STING agonist can be administered each time the anti-CTLA4 antibody is administered. Alternatively, the STING agonist can be administered more frequently than the anti-CTLA4 antibody. For instance, STING agonist can be administered weekly or biweekly, and the anti-CTLA4 antibody can be administered biweekly, triweekly, every four weeks, or monthly.

In embodiments relating to administration of a CTLA4 inhibitor, a STING agonist and a PD-1 (or PD-L1) inhibitor, the particular doses and dosing schedule of the CTLA4 inhibitor and PD-1 (or PD-L1) inhibitor will depend on the particular inhibitor and the cancer being treated. In embodiments where the CTLA4 and PD-1 (or PD-L1) inhibitors are antibodies, the antibodies may be delivered according to the same dosing schedule or on alternative dosing schedules. In one embodiment, the STING agonist and the anti-CTLA4 antibody can be administered intratumorally according to a particular dosing schedule and the anti-PD-1 antibody (or anti-PD-L1 antibody) can be administered systemically (e.g., intravenously, subcutaneously, or intramuscularly) on an alternative dosing schedule. In one such embodiment, the anti-CTLA4 antibody and the STING agonist can be administered conjointly and intratumorally on a weekly, biweekly, or triweekly schedule for a particular number of doses, which is followed by administration of the anti-PD-1 antibody (or anti-PD-L1 antibody) every 2-4 weeks for the remainder of the dosing schedule.

In one embodiment, the anti-CTLA4 antibody is ipilimumab, and the ipilimumab and the STING agonist are both administered intratumorally to the cancer patient. With respect to ipilimumab, the intratumoral dose can vary between 0.01 mg/kg to 1 mg/kg. For instance, the intratumoral dose of ipilimumab can vary between 0.01 mg to 0.5 mg/kg, 0.05 mg to 0.5 mg/kg, 0.1 mg/kg to 0.5 mg/kg, 0.2 mg/kg to 0.5 mg/kg, 0.2 mg/kg to 0.4 mg/kg, 0.2 mg/kg to 0.3 mg/kg. In particular embodiments, ipilimumab and the STING agonist can be conjointly administered weekly, biweekly, or triweekly. In other embodiments, the STING agonist can be administered weekly and ipilimumab can be administered biweekly. In other embodiments, the STING agonist can be administered weekly or biweekly and ipilimumab can be administered triweekly. In other embodiments, the STING agonist can be administered weekly or biweekly and ipilimumab can be administered every 4 weeks or monthly. In other embodiments, the STING agonist can be administered according to dosing schedules discussed herein, such as weekly for the first three weeks for a first 28-day cycle and biweekly in subsequent cycles, and ipilimumab can be administered biweekly in all cycles. In one particular embodiment, the STING agonist administered in combination with ipilimumab is Compound A. In this embodiment, Compound A can be administered via a dosing regimen described in Section 5.5.

In another embodiment, the anti-CTLA4 antibody ipilimumab and the STING agonist are both administered intratumorally to the cancer patient in combination with an anti-PD-1 antibody or anti-PD-L1 antibody. The anti-PD-1 antibody or anti-PD-L1 antibody can be administered on the same dosing schedule or on an alternative dosing schedule as the ipilimumab and the STING agonist. In one embodiment, ipilimumab and the STING agonist are administered conjointly and intratumorally in accordance with a dosing schedule set forth in the preceding paragraph and the anti-PD-1 antibody or anti-PD-L1 antibody is administered systemically (e.g., intravenously, subcutaneously, or intramuscularly) subsequent to the completion of the intratumoral dosing regimen. For instance, ipilimumab and the STING agonist can be administered to the cancer patient conjointly and intratumorally every 2-3 weeks for 4-8 doses, followed by administration of the anti-PD-1 antibody or anti-PD-L1 antibody every 2-4 weeks for the duration of the treatment. In one particular embodiment, the STING agonist administered in combination with ipilimumab is Compound A. In this embodiment, Compound A can be administered via a dosing regimen described in Section 5.5.

The dosage of the STING agonist will vary depending on the particular STING agonist and the route of administration. In general, for systemic or intratumoral administration, the STING agonist can be administered at a dose in the range of 1-1000 μg/kg. For oral administration, the STING agonist can be administered at a dose in the range of 5-5000 μg/kg.

5.5. STING Agonist Dosing Regimens with Improved Safety Profiles

In some embodiments, the STING agonist is administered under a dosing schedule that includes a priming dose followed by multiple maintenance doses. A priming dose refers to a dose that is administered at lower doses than the maintenance doses to increase the tolerance of the body for a particular active agent (e.g., a STING agonist). It has been found that administration of a priming dose of the STING agonist improves the safety profile of the STING agonist and allows the compound to be delivered at higher maintenance dosage levels than would otherwise be tolerated. In general, the priming dosage amount will be less than the maintenance doses over the course of a given dosing cycle.

Accordingly, the disclosure provides novel dosing schedules for STING agonists based on specific dosing schedules requiring administration of a priming dose followed by administration of maintenance doses. The STING agonist can be administered by itself or in combination with one or more anti-cancer agents. The STING agonist can be administered intratumorally, systemically or orally. In particular embodiments, the novel STING agonist dosing schedules described herein also involve conjoint administration with one or more immune checkpoint inhibitors, particularly a CTLA4 inhibitor, PD-1 inhibitor, or a PD-L1 inhibitor. In particular embodiments, the CTLA4, PD-1 and PD-L1 inhibitors conjointly administered with the STING agonist are described in Section 5.1, In particular embodiments, the CTLA4 inhibitor is administered intratumorally, as described herein, including in Sections 5.1 to 5.2. Using the combination of the STING agonist priming/maintenance dosing regimen conjointly with intratumoral CTLA4 dosing is expected to provide an improved therapeutic index.

Particular STING agonists that can be administered using the disclosed priming/maintenance dosing schedules are described in Section 5.1, above. In some embodiments, the STING agonist to be administered with the disclosed priming/maintenance dosing schedule is Compound A. In some embodiments, the STING agonist to be administered with the disclosed priming/maintenance dosing schedule is not Compound A. In some embodiments, the STING agonist to be administered with the disclosed priming/maintenance dosing schedule is Compound B. In some embodiments, the STING agonist to be administered with the disclosed priming/maintenance dosing schedule is Compound C. In some embodiments, the STING agonist to be administered with the disclosed priming/maintenance dosing schedule is Compound D. In some embodiments, the STING agonist to be administered with the disclosed priming/maintenance dosing schedule is Compound E. In some embodiments, the STING agonist to be administered with the disclosed priming/maintenance dosing schedule is Compound F. In some embodiments, the STING agonist to be administered with the disclosed priming/maintenance dosing schedule is Compound G. In certain embodiments, the STING agonist to be administered with the disclosed priming/maintenance dosing schedule is administered as part of an ADC, such as those described herein.

In some embodiments, the priming dose of the STING agonist can be administered in a quantity (by weight) that is 2- to 100-fold less than the individual maintenance doses in a given dosing cycle. For instance, the priming dose can be administered in a quantity that is 2- to 70-fold less than, 2- to 50-fold less than, 2- to 30-fold less than, 2- to 20-fold less than, 2- to 10-fold less than, 10- to 50-fold less than, 10- to 30-fold less than, 10- to 20-fold less, or 20- to 30-fold less than the maintenance doses in a given cycle. In some embodiments, the priming dose can be administered in a quantity that is 2- to 4-fold less than the maintenance doses in a given cycle. In some embodiments, the priming dose can be administered in a quantity that is 2- to 5-fold less than the maintenance doses in a given cycle. In some embodiments, the priming dose can be administered in a quantity that is 2- to 8-fold less than the maintenance doses in a given cycle. In some embodiments, the priming dose can be administered in a quantity that is 3- to 5-fold less than the maintenance doses in a given cycle. In some embodiments, the priming dose can be administered in a quantity that is 3- to 8-fold less than the maintenance doses in a given cycle. In some embodiments, the priming dose can be administered in a quantity that is 4- to 8-fold less than the maintenance doses in a given cycle.

In some embodiments, the priming dose can be delivered at a dose that is about 2-fold less than the maintenance doses over the course of a dosing cycle. In some embodiments, the priming dose can be delivered at a dose that is about 3-fold less than the maintenance doses over the course of a dosing cycle. In some embodiments, the priming dose can be delivered at a dose that is about 4-fold less than the maintenance doses over the course of a dosing cycle. In some embodiments, the priming dose can be delivered at a dose that is about 5-fold less than the maintenance doses over the course of a dosing cycle. In some embodiments, the priming dose can be delivered at a dose that is about 10-fold less than the maintenance doses over the course of a dosing cycle. In some embodiments, the priming dose can be delivered at a dose that is about 15-fold less than the maintenance doses over the course of a dosing cycle. In some embodiments, the priming dose can be delivered at a dose that is about 20-fold less than the maintenance doses over the course of a dosing cycle. In some embodiments, the priming dose can be delivered at a dose that is about 50-fold less than the maintenance doses over the course of a dosing cycle. In some embodiments, the priming dose can be delivered at a dose that is about 100-fold less than the maintenance doses over the course of a dosing cycle.

It should be understood that the above relative amounts of priming dose to the individual maintenance doses can be expressed as a ratio. For instance, in an embodiment where the priming dose is administered at a dose that is about 2-fold less than the maintenance doses, a dosing regimen that involves a 1:2 ratio of priming dose to individual maintenance doses is described. Accordingly, in certain embodiments, the present disclosure provides a method of treating cancer comprising administering the STING agonist to a patient in need thereof according to a dosing regimen that includes a 1:2 to 1:100 ratio of priming dose to individual maintenance doses, such as a ratio of 1:2, 2:5, 3:8, 1:3, 2:7, 1:4, 1:5, 1:6, 1:8, 1:9, 1:10, 1:11, 1:12, 1:15, 1:20, 1:30, 1:50, 1:75, or 1:100, including ranges created by these ratios, such as 1:2 to 1:3, 1:2 to 1:4, 1:2 to 1:5, 1:2 to 1:8, 1:2 to 1:10, 1:4 to 1:8, 1:4 to 1:10, 1:4 to 1:15, 1:4 to 1:20, 1:8 to 1:10, 1:8 to 1:15, 1:8 to 1:20, 1:8 to 1:30, 1:10 to 1:15, 1:10 to 1:20, 1:10 to 1:30, 1:10 to 1:50, 1:20 to 1:30, 1:20 to 1:50, 1:20 to 1:75, 1:20 to 1:100, 1:30: to 1:50, 1:30 to 1:75, 1:30 to 1:100, 1:50 to 1:75, 1:50 to 1:100, or 1:75 to 1:100.

In some embodiments, the present disclosure provides a method of treating cancer comprising administering the STING agonist to a patient in need thereof according to a dosing regimen that includes a 1:4 or 1:5 ratio of priming dose to individual maintenance doses, or a ratio in the range of 1:3 to 1:6, such as 1:3 to 1:5, 1:4 to 1:6, or 1:4 to 1:5. In other embodiments, the ratio is 1:8 or 1:10, or a ratio in the range of 1:5 to 1:15, such as 1:6 to 1:12, 1:8 to 1:12, 1:8 to 1:10, or 1:9 to 1:10.

In some embodiments, the priming dose can be administered on day 1 of a treatment cycle and the maintenance doses can be administered thereafter at a dosing schedule as described above. The first maintenance dose can be administered at least 2 days following the administration of the priming dose, i.e., on day 3. For instance, the first maintenance dose can be administered 2, 3 4, 5, 6, 7, 8, 9, or 10 days following administration of the priming dose.

In one embodiment, the dosing cycle comprises administering a priming dose of the STING agonist on day 1 of a treatment cycle followed by administering maintenance doses of the STING agonist on days 8, 15 and 22 (i.e., the first day of weeks 2, 3 and 4) of the treatment cycle, followed by a period of one week (i.e., week 5) where the STING agonist is not administered to the patient. The maintenance dosing cycle can be repeated or a modified maintenance dosing schedule can be employed.

In another embodiment, the dosing cycle comprises administering a priming dose of the priming dose on day 1 of a treatment cycle followed by administering maintenance doses of the STING agonist on days 8 and 22 of the dosing schedule (i.e., biweekly dosing). The maintenance dosing cycle can be repeated or a modified maintenance dosing schedule can be employed.

5.6. Intratumoral CTLA4 Inhibitor in Combination with Compound A

While intratumoral administration improves the therapeutic index of the CTLA4 inhibitor, the choice of a particular STING agonist can further improve the safety profile. Ideally, the STING agonist evokes a powerful anti-tumor effect with significantly reduced concurrent side effects often associated with excessive cytokine production. It has been found that Compound A is a STING agonist that is capable of eliciting the production of cytokines in a dose dependent manner. Compound A exhibits a profound anti-tumor effect, even at very low levels of cytokine production. For instance, Compound A can be administered safely to cancer patients and provide therapeutic benefits when administered in the range of 1-100 μg/kg. When compound A is administered conjointly with an intratumoral dosage of a CTLA4 inhibitor, in accordance with the present disclosure, a significantly improved therapeutic index is achieved.

In particular embodiments where Compound A serves as the STING agonist, Compound A can be administered intratumorally or systemically in the range of 1-100 μg/kg. For instance, Compound A can be administered to a cancer patient in the range of 1-10 μg/kg, 5-10 μg/kg, 5-20 μg/kg, 5-30 μg/kg, 5-40 μg/kg, 5-50 μg/kg, 10-20 μg/kg, 10-30 μg/kg, 10-40 μg/kg, 10-50 μg/kg, 15-20 μg/kg, 15-40 μg/kg, 20-30 μg/kg, 20-40 μg/kg, 20-50 μg/kg, 30-40 μg/kg, 30-50 μg/kg, 5-75 μg/kg, 10-75 μg/kg, 15-75 μg/kg, 20-75 μg/kg, 25-75 μg/kg, 35-75 μg/kg, 5-100 μg/kg, 10-100 μg/kg, 15-100 μg/kg, 20-100 μg/kg, 25-100 μg/kg, 35-100 μg/kg, or 50-100 μg/kg.

In some embodiments, Compound A can be administered to a cancer patient at a dose, e.g., a single or divided doses, in the range of 10-6,500 μg, such as 50-6,500 μg. In particular embodiments, Compound A can be administered to a cancer patient at a dosage, e.g., a single or divided doses, in the range of 100-3,000 μg. In other embodiments, Compound A can be administered to a cancer patient at a dosage e.g., a single or divided doses, in the range of 100-1,200 μg. For instance, Compound A can be administered to a cancer patient in the range of 10-50 μg, 10-100 μg, 10-200 μg, 50-200 μg, 100-200 μg, 100-400 μg, 100-500 μg, 100-800 μg, 200-400 μg, 400-600 μg, 400-800 μg, 100-1,000 μg, 250-1,000 μg, 500-1,000 μg, 500-3,000 μg, 1,000-3,000 μg, 500-4,500 μg, 1,000-4,500 μg, 500-6,500 μg, 1,000-6,500 μg, 2,000-6,500 μg, 3,000-6,500 μg, or 4,500-6,500 μg.

In embodiments involving the administration of priming and maintenance doses of Compound A, the priming dose of Compound A can be administered to a cancer patient at a dosage in the range of 10-1,000 μg. For instance, the priming dose of Compound A can be administered to a cancer patient in the range of 10-20 μg, 10-40 μg, 10-50 μg, 10-80 μg, 20-40 μg, 40-60 μg, 40-80 μg, 50-100 μg, 100-200 μg, 100-300 μg, 100-500 μg, 200-500 μg, 200-800 μg, 200-1,000 μg, 500-800 μg, or 500-1,000 μg. In certain embodiments, the priming dose of Compound A can be administered to a cancer patient at a dosage in the range of 0.15-20 μg/kg, such as 0.15-1 μg/kg, 0.25-1 μg/kg, 0.5-1 μg/kg, 0.5-2 μg/kg, 1-3 μg/kg, 1-5 μg/kg, 2-5 μg/kg, 2-7 μg/kg, 1-10 μg/kg, 2-10 μg/kg, 3-10 μg/kg, 5-10 μg/kg, 5-15 μg/kg, 10-20 μg/kg, or 15-20 μg/kg.

In embodiments involving the administration of priming and maintenance doses of Compound A, the maintenance dose of Compound A can be administered to a cancer patient at a dosage in the range of 100-3,000 μg. In other embodiments, the maintenance doses of Compound A can be administered to a cancer patient at a dosage in the range of 100-1,200 μg. For instance, the maintenance doses of Compound A can be administered to a cancer patient in the range of 50-200 μg, 100-200 μg, 100-400 μg, 100-500 μg, 100-800 μg, 100-1,000 μg, 200-400 μg, 200-800 μg, 200-1,200 μg, 250-1,000 μg, 400-600 μg, 400-800 μg, 400-1,200 μg, 500-1,000 μg, 500-1,200 μg, 500-1,500 μg, 500-2,000 μg, 500-4,500 μg, 800-1,200 μg, 800-1,500 μg, 800-2,000 μg 1,000-2,000 μg, 1,000-3,000 μg, 1,000-4,500 μg, 2,000-4,500 μg, 500-6,500 μg, 1,000-6,500 μg, 1,500-6,500 μg, 2,000-6,500 μg, or 3,000-6,500 μg. In certain embodiments, the maintenance doses of Compound A can be administered to a cancer patient at a dosage in the range of 1-100 μg/kg, such as 1-50 μg/kg. For instance, the maintenance doses of Compound A can be administered to a cancer patient in the range of 1-10 μg/kg, 5-10 μg/kg, 5-20 μg/kg, 5-30 μg/kg, 5-40 μg/kg, 5-50 μg/kg, 10-20 μg/kg, 10-30 μg/kg, 10-40 μg/kg, 10-50 μg/kg, 15-20 μg/kg, 15-40 μg/kg, 20-30 μg/kg, 20-40 μg/kg, 20-50 μg/kg, 30-40 μg/kg, 30-50 μg/kg, 5-75 μg/kg, 10-75 μg/kg, 15-75 μg/kg, 20-75 μg/kg, 25-75 μg/kg, 35-75 μg/kg, 5-100 μg/kg, 10-100 μg/kg, 15-100 μg/kg, 20-100 μg/kg, 25-100 μg/kg, 35-100 μg/kg, or 50-100 μg/kg.

In another embodiment, the dosing cycle comprises administering a priming dose of Compound A on day 1 of a treatment cycle followed by administering Compound A under two maintenance dosing regimens. The first maintenance dosing regimen comprises administering maintenance doses Compound A on days 8, 15 and 22 (i.e., the first day of weeks 2, 3 and 4) of the treatment cycle, followed by a period of one week (i.e., week 5) where Compound A is not administered to the patient. The second maintenance dosing regimen comprises administering Compound A on a biweekly dosing regimen. For instance, Compound A can be administered at the beginning of weeks 6 and 8 of the dosing cycle. In some embodiments, additional biweekly dosing of Compound A can be administered to the patient. For instance, Compound A can be administered at week 10 of the dosing cycle, weeks 10 and 12 of the dosing cycle, weeks 10, 12, and 14 of the dosing cycle, weeks 10, 12, 14, and 16 of the dosing cycle, and so on.

### 6. EXAMPLES

### Example 1. Administering Priming and Maintenance Doses of Compound A

Male and female cynomolgus monkeys were assigned to groups and doses of Compound A were administered. Animals were dosed via subcutaneous injection at a volume of 2 mL/kg. The vehicle control article/diluent was phosphate-buffered saline (PBS).

Escalation of Compound A dose levels was tolerated up to 3.0 mg/kg/dose, with findings limited to increased body temperature and elevated IFNα, IL-6, and TNFα cytokine levels. IFNα, TNFα, and IL-6 levels were measured at 3, 6, and 12 hours post-dosing. Dose related but variable changes were observed. Moderate levels of IFNα were noted in the 1 mg/kg and 3 mg/kg groups at 3 hours and 6 hours post dosing. Higher levels of IFNα were seen in the 10 mg/kg group. IFNα levels at 3 mg/kg and 10 mg/kg decreased 12 hours after dosing, but did not return to pre-dose levels. Increases in plasma IL-6 levels were noted at 3 and 6 hours post dosing in all groups. IL-6 increases at 3 mg/kg and 10 mg/kg persisted at 12 hours postdose. TNFα levels increased at 3 hours in the 1 mg/kg group. Lower levels of TNFα were observed in the 3 mg/kg and 10 mg/kg groups. The cytokine responses are consistent with the predicted STING pathway activation. Morbidity was observed within 1 day of administration of the 10 mg/kg/dose; as such, 3 mg/kg was selected as the high dose for the following repeat-dose phase (Phase II).

In Phase II, 3 weekly administrations of 0.3 mg/kg of Compound A were tolerated. The 3 mg/kg dose in naïve animals was not tolerated and led to clinical observations of morbidity or death within 1 day of dosing. The findings were consistent with Compound A-mediated inflammatory response that was considered the probable cause of death. At the 3 mg/kg dose level, compound-related dose-dependent increases in plasma IL-1ra, IL-6, and IFNα cytokine levels were generally noted at 3 and 6 hours with levels returning to those noted in controls for IL-6 and IFNα. There were sporadic increases in IL-12, granulocyte-colony stimulating factor (G-CSF), and IFNγ levels. These changes, however, were generally inconsistent between sexes, not dose-dependent, and of a small magnitude and, hence, considered only potentially related to Compound A. Changes in levels of pro-inflammatory cytokines and chemokines MCP-1 and IP-10 were suggestive of an inflammatory response with resolution by 24 hours postdose. Exposure, as assessed by Compound A mean Cₘₐₓ, AUC₀₋₂, AUC₀₋₈, and AUC₀₋₂₄ values, generally increased with the increase in dose level from 0.3 to 3 mg/kg/day on Day 1 of Phase II, and were generally dose-proportional. No accumulation of Compound A was observed after multiple doses of 0.3 mg/kg/day in monkeys. In general, sex differences in Compound A mean Cₘₐₓ, AUC₀₋₂, AUC₀₋₈, and AUC₀₋₂₄ values were less than 2-fold.

During Phase III, all animals administered three weekly doses of 0.6 or 1.0 mg/kg/day of Compound A survived until scheduled sacrifice. A priming dose of 0.1 mg/kg/day was administered 4 days prior to the first dose of 1.0 mg/kg/day Compound A to potentially allow a tolerance to develop to avoid the acute mortality noted during Phase II following administration of 3.0 mg/kg/day of Compound A to naïve animals. When administered at 0.1 mg/kg/day, Compound A did not cause significant increase in plasma IFNα levels in either male or female. Increased plasma levels of IL-6 were noted 3 hours and 6 hours postdose; however, IL-6 levels returned to a non-detectable level 24 hours postdose. Elevated levels of TNFα were noted 6 hours postdose in male and 3 hours and 6 hours postdose in female. In both cases, TNFα levels returned to non-detectable level 24 hours post dosing. Slight elevation of IP-10 was noted 3 hours post dosing in male and female animals. When administered at 0.6 mg/kg/day, Compound A did not cause significant increase in plasma IFNα levels in either male or female. Increased plasma levels of IL-6 were noted 3 hours and 6 hours postdose. Elevated levels of TNFα were noted 6 hours postdose in male and 1.5, 3, and 6 hours postdose in female. No significant elevation of IP-10 was noted throughout the time course. When administered at 1 mg/kg/day, Compound A did not cause significant change in IFNα levels at 1.5 and 3 hours postdose, but elevated levels of this cytokine were observed 6 hours postdose in both male and female. Marked increase in IL-6 levels was noted at 3 and 6 hours postdose in both male and female. Elevated TNFα levels were noted at 1.5, 3, and 6 hours postdose in both male and female. A slightly higher predose level of IP-10 was noted in male only, but no increased IP-10 level was observed 1.5, 3, and 6 hours postdose.

In conclusion, administration of ≥3.0 mg/kg of Compound A was not tolerated in naïve animals and led to acute morbidity and/or death, which was attributed to pulmonary edema. Edema is consistent with an inflammatory related pathology and the exaggerated pharmacology of the mode of action of Compound A. Administration of 3 weekly doses of 1.0 mg/kg/day (preceded by a priming dose of 0.1 mg/kg) or 0.6 mg/kg (without a priming dose) was tolerated. Animals tolerated an escalation to 3.0 mg/kg in Phase I, due to previous administrations at lower levels that allowed a tolerance to develop. For animals administered with 0.6 or 1.0 mg/kg/day, compound-related findings were limited to a transient body temperature increase and mild to moderate clinical and anatomic pathology findings.

### Example 2. Combination Studies

Anti-CTLA4 antibody therapy is an FDA-approved immune checkpoint blockade therapy. However, systemic administration of this antibody is often associated with considerable toxicity. Intratumoral injection of an anti-CTLA4 antibody conjointly with Compound A was examined.

On day 0, female C57BL6 mice (5 in each group) were subcutaneously implanted with 10⁶ of B16F10 melanoma cells (ATCC CRL6475) on their flanks. On day 6, tumors were measured and mice were regrouped so that each group had similar average tumor volumes (˜70 mm³). On day 6, 10, and 14, mice were mock treated or treated with: 0.3 μg of Compound A intratumorally (I.T.); 50 μg of anti-CTLA4 antibody (BioXcell BE0164, I.T.); combination of 0.3 μg of Compound A and 10 μg of anti-CTLA4 antibody (both I.T.); combination of 0.3 μg of Compound A and 50 μg of anti-CTLA4 antibody (both I.T.); or combination of 0.3 μg of Compound A (I.T.) and 200 μg of anti-CTLA4 antibody intraperitoneally (I.P.). In the same set of experiments, the combination of 0.3 μg of Compound A (I.T.) and 200 μg of anti-PD-L1 antibody (I.P.) was also tested with and without the combination of 200 μg of anti-CTLA4 antibody (I.P.). Tumor volumes were measured every 2-3 days and mouse survival was monitored daily.

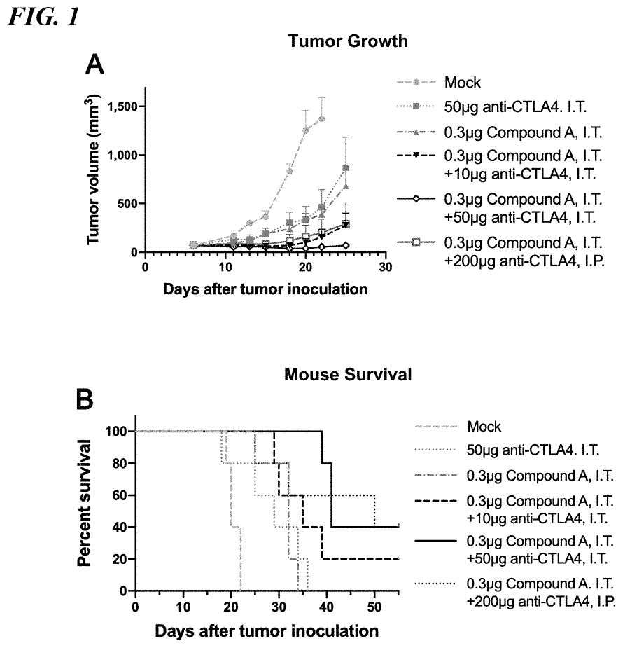

Intratumoral administration of either 50 μg of anti-CTLA4 antibody or 0.3 μg of Compound A alone reduced tumor growth and extended mouse survival to comparable extents (FIG. 1, panels A and B). Combining 10 μg of anti-CTLA4 antibody (I.T.) with 0.3 μg of Compound A (I.T.) further suppressed tumor growth and improved mouse survival compared to both Compound A alone and anti-CTLA4 antibody alone. Increasing anti-CTLA4 antibody (I.T.) in the combination treatment to 50 μg led to more dramatic tumor remission. This combination treatment was more effective than combining of 0.3 μg of Compound A (I.T.) with 200 μg of anti-CTLA4 antibody (I.P.) (FIG. 1, panel A). When combining anti-CTLA4 antibody therapy with Compound A, the intratumoral route for anti-CTLA4 antibody at a lower dose of anti-CTLA4 antibody was superior to the systemic route at a higher dose of anti-CTLA4 antibody. Specifically, the combination treatment of 0.3 μg of Compound A (I.T.) with 50 μg of anti-CTLA4 antibody (I.T.) was more effective in suppressing the tumor growth than the combination of 0.3 μg of Compound A (I.T.) with 200 μg of anti-CTLA4 antibody (I.P.) (FIG. 1, panel A).

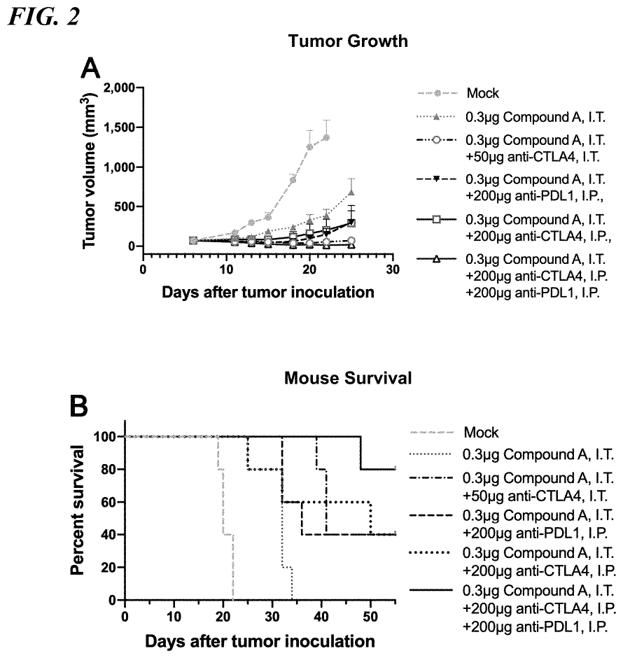

The above-noted effect of 0.3 μg of Compound A (I.T.) with 50 μg of anti-CTLA4 antibody (I.T.) on tumor growth was comparable to the triple combination of 0.3 μg Compound A (I.T.), 200 μg anti-PD-L1 antibody (I.P.), and 200 μg anti-CTLA4 antibody (I.P.) (FIG. 2, panel A). The combinations of 0.3 μg of Compound A (I.T.) with 200 μg of anti-CTLA4 antibody (I.P.) and 0.3 μg of Compound A (I.T.) with 200 μg of anti-PD-L1 antibody (I.P.) had similar reductions in tumor growth, but both were inferior to 0.3 μg of Compound A (I.T.) with 50 μg of anti-CTLA4 antibody (I.T.) (FIG. 2, panel A), and these three combinations had similar survival benefits FIG. 2, panel B).

### Example 3. Further Combination Studies

Intratumoral injection of an anti-CTLA4 antibody conjointly with the STING agonist DMXAA was examined.

Female C57BL6 mice at the age of 7-8 weeks were implanted on day 0 with 10⁶ of B16F10 melanoma cells (ATCC CRL-6475) subcutaneously on their right flanks. On day 6, tumors were measured and mice were regrouped so that each group (n=5) had similar average tumor volumes (˜120 mm³). On day 6, 9, 12, 15, mice were treated intratumorally with 50 μg of anti-CTLA4 antibody (BioXcell, BE0614), or 50 μg of DMXAA (Sigma-Aldrich, D5817), or the combination of both the anti-CTLA4 antibody and DMXAA. Mock treated group were injected with PBS intratumorally. Tumor volumes were measured every 2-3 days and mouse survival was monitored daily.

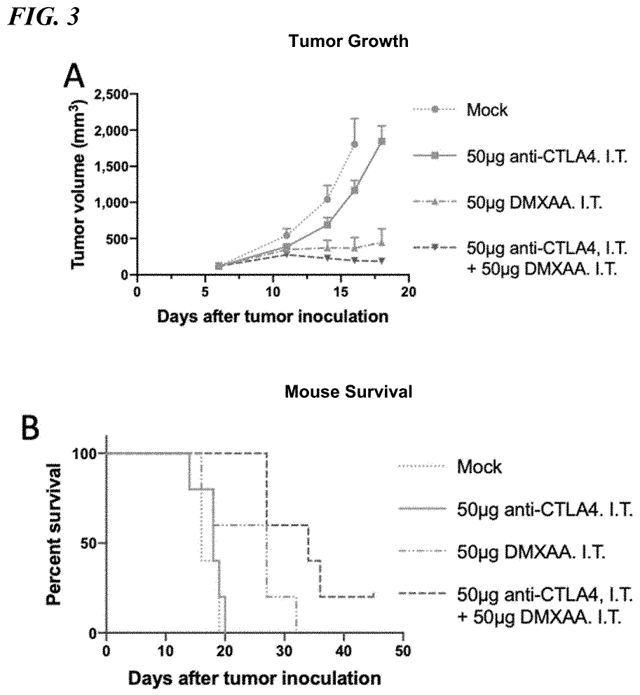

Treatment with the anti-CTLA4 antibody alone partially reduced tumor growth rate but had no effect on mice survival. DMXAA alone greatly suppressed tumor growth, and prolonged survival. However, the combination of anti-CTLA4 antibody and DMXAA showed significant improved effect compared to monotherapy with either the anti-CTLA4 antibody or DMXAA in controlling tumor growth (FIG. 3, panel A) and extending survival (FIG. 3, panel B).

1. A method of treating a cancer in a patient in need thereof, comprising conjointly administering a CTLA4 inhibitor and a STING agonist to the patient, wherein the CTLA4 inhibitor is administered intratumorally.

2. The method of claim 1, wherein the CTLA4 inhibitor is an anti-CTLA4 antibody.

3. (canceled)

4. The method of claim 1, wherein the STING agonist is administered intratumorally.

5. The method of claim 1, wherein the STING agonist is administered systemically.

6. The method of claim 5, wherein the STING agonist is administered intravenously.

7-8. (canceled)

9. The method of claim 1, wherein the STING agonist is a cyclic dinucleotide.

10. The method of claim 9, wherein the cyclic dinucleotide has the following structure or a pharmaceutically acceptable salt thereof:

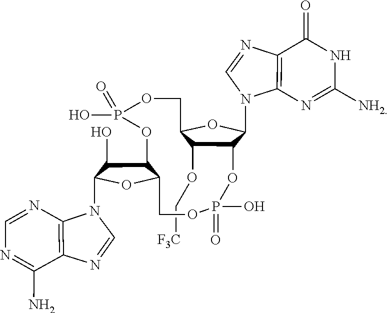

11. The method of claim 1, wherein the STING agonist is administered orally.

12. The method of claim 1, further comprising administering a PD-1 inhibitor to the patient.

13. (canceled)

14. The method of claim 12, wherein the PD-1 inhibitor is administered systemically to the patient.

15. The method of claim 14, wherein the PD-1 inhibitor is administered intravenously, subcutaneously, or intramuscularly to the patient.

16. The method of claim 12, wherein the PD-1 inhibitor is administered intratumorally to the patient.

17. The method of claim 1, further comprising administering a PD-L1 inhibitor to the patient.

18. (canceled)

19. The method of claim 17, wherein the PD-L1 inhibitor is administered systemically to the patient.

20. The method of claim 19, wherein the PD-L1 inhibitor is administered intravenously, subcutaneously, or intramuscularly to the patient.

21. The method of claim 17, wherein the PD-L1 inhibitor is administered intratumorally to the patient.

22. (canceled)

23. A method of augmenting the anti-tumor response of a CTLA4 inhibitor administered intratumorally to a cancer patient, comprising conjointly administering a STING agonist and the CTLA4 inhibitor to the patient.

24-44. (canceled)

45. A pharmaceutical composition for intratumoral injection, comprising a CTLA4 inhibitor, a STING agonist, and a pharmaceutically acceptable carrier.

46-51. (canceled)

52. A method of treating a cancer in a patient in need thereof, comprising administering to the patient a STING agonist according to a dosing regimen comprising a priming dose of the STING agonist followed by maintenance doses of the STING agonist, wherein the amount of the STING agonist in the priming dose is less than the amount of the STING agonist in each maintenance dose.

53-68. (canceled)

69. The method of claim 1, wherein the STING agonist is selected from ADU-S100 (MIW815), BMS-986301, CRD5500, CMA (10-carboxymethyl-9-acridanone), diABZI STING agonist-1 (e.g., CAS No.: 2138299-34-8), DMXAA (ASA404/vadimezan), E7766, GSK-532, GSK-3745417, MK-1454, MK-2118, SB-11285, SRCB-0074, TAK-676, TTI-10001, SR-717, and MSA-2.

70-77. (canceled)
